(19) United States
(12) Patent Application Publication
(10) Pub. No.: US 2014/0179557 A1
(43) Pub. Date: Jun. 26, 2014

(54) MARKER SEQUENCES FOR DIAGNOSING PROSTATE CANCER, AND USE THEREOF

(71) Applicant: PROTAGEN AKTIENGESELLSCHAFT, DORTMUND (DE)

(72) Inventors: ANGELIKA LUEKING, BOCHUM (DE); AXEL KOWALD, BOCHUM (DE); HELMUT KLOCKER, INZING (AT)

(73) Assignee: PROTAGEN AKTIENGESELLSCHAFT, DORTUND (DE)

(21) Appl. No.: 14/188,425

(22) Filed: Feb. 24, 2014

Related U.S. Application Data
(63) Continuation of application No. 13/985,218.

(30) Foreign Application Priority Data
Feb. 13, 2011 (EP) 11154261.9

(51) Int. Cl. G01N 33/574

(52) U.S. Cl. CPC G01N 33/57434
USPC 506/9; 506/18

(57) ABSTRACT

The present invention relates to novel marker sequences for prostate carcinoma while excluding inflammatory prostate diseases or diabetes or polymorbidity and relates to their diagnostic use including a method for screening potential active substances for such prostate diseases by means of these marker sequences. The invention furthermore relates to a diagnostic device including such marker sequences for prostate carcinoma, in particular a protein biochip and its use.

### RELATED APPLICATIONS

This application is a continuation application of application Ser. No. 13/985,218 filed Aug. 13, 2013, which is a national stage application (under 35 U.S.C. §371) of PCT/EP2012/052438, filed Feb. 13, 2012, which claims priority to European Application No. 11154261.9, filed Feb. 13, 2011. The entire contents of each of these applications are hereby incorporated by reference herein in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is US_Sequence_Listing. The size of the text file is 2,096 KB, and the text file was created on Feb. 24, 2014.

### DESCRIPTION

The present invention relates to novel marker sequences for prostate carcinoma (syn.: prostate cancer) while excluding inflammatory prostate diseases, diabetes, and polymorbidity, and relates to the diagnostic use thereof together with a method for screening potential active substances for prostate diseases of this type by means of these marker sequences. Furthermore, the method relates to a diagnostic device containing marker sequences of this type for prostate carcinoma, in particular a protein biochip and the use thereof.

Protein biochips are gaining increasing industrial importance in analysis and diagnosis as well as in pharmaceutical development. Protein biochips have become established as screening instruments.

The rapid and highly parallel detection of a multiplicity of specifically binding analysis molecules in a single experiment is rendered possible hereby. To produce protein biochips, it is necessary to have the required proteins available. For this purpose, in particular protein expression libraries have become established. The high throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P., (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Reboul, J., Vaglio, P., Rual, J. F., Lamesch, P., Martinez, M., Armstrong, C M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome Version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, an approach of this type is strongly connected to the progress of the genome sequencing projects and the annotation of these gene sequences. Furthermore, the determination of the expressed sequence can be ambiguous due to differential splicing processes. This problem may be circumvented by the application of cDNA expression libraries (Bussow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C, Lehrach, H. and Walter, G. (2000) A human cDNA library for high-throughput protein expression screening. Genomics, 65, 1-8; Holz, C, Lueking, A., Bovekamp, L., Gutjahr, C, Bolotina, N., Lehrach, H. and Cahill, D. J. (2001) A human cDNA expression library in yeast enriched for open reading frames. Genome Res, 11, 1730-1735; Lueking, A., Holz, C, Gotthold, C, Lehrach, H. and Cahill, D. (2000) A system for dual protein expression in Pichia pastoris and Escherichia coli, Protein Expr. Purif., 20, 372-378). The cDNA of a particular tissue is hereby cloned into a bacterial or an eukaryotic expression vector, such as, e.g., yeast. The vectors used for the expression are generally characterized in that they carry inducible promoters that may be used to control the time of protein expression. Furthermore, expression vectors have sequences for so-called affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

For example, the gene products of a cDNA expression library from human fetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from the library could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Büssow (2000) supra; Lueking, A., Horn, M., Eickhoff, H., Büssow, K., Lehrach, H. and Walter, G. (1999) Protein microarrays for gene expression and antibody screening. Analytical Biochemistry, 270, 103-111). Protein biochips of this type based on cDNA expression libraries are in particular the subject matter of WO 99/57311 and WO 99/57312.

Furthermore, in addition to antigen-presenting protein biochips, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

However, there is a great need to provide indication-specific diagnostic devices, such as a protein biochip.

The laboratory parameters for the diagnosis of prostate carcinoma include acid phosphatase (AP) and prostate-specific antigen (PSA). Above all, PSA currently has a high importance in diagnostics. It is specific for the prostate, but not for a tumor disease, but rather can also be elevated in the event of inflammation, benign prostate hyperplasia, urine retention, or without an obvious reason. A value over 4 ng/mL already requires clarification.

Applicant's WO2010/000874 already describes the diagnosis of prostate carcinoma and prostate inflammations by means of a protein biochip and provides certain diagnostic marker sequences.

However, it is a disadvantage that these markers are also suitable for the diagnosis of prostate inflammation so that it is not possible to discriminate within the patient group of prostate cancer patients with these marker sequences that are known in the prior art. It is furthermore a drawback of the prior art that false-positive marker sequences may be identified based on other indications such as prostate inflammation and diabetes.

WO 2009/080017 discloses marker sequences for neurodegenerative diseases and the use thereof. The sequences disclosed in WO 2009/080017 were obtained accordingly using a method without the use of samples from prostate cancer patients.

US 2004/259086 relates to compositions, kits, and methods for detecting, characterizing, preventing, and treating human prostate cancers. A variety of marker sequences were used whose levels of expression correlate to prostate cancer. however, US 2004/259086 does not disclose any marker sequences for the diagnosis of prostate carcinoma while excluding inflammatory prostate diseases, diabetes, and polymorbidity. The marker sequences in US 2004/259086 were obtained using subtraction of cDNA libraries produced from the mRNA of patients with benign prostatic hyperplasia and the cDNA produced from the mRNA of patients with prostate cancer ([0339] ff. in US 2004/259086). There was no further validation. These marker sequences are thus not suitable for identifying patient subgroups within the group of prostate cancer patients.

DNA sample arrays HG-U95, HG-U133 and HG-U133 (Affymetrix) disclose the SEQ ID Nos. 1 and 247 already used in the present application. DNA sample arrays HG-U95, HG-U133 and HG-U133 were not validated for specific diagnostic uses, however.

The object of the present invention is to provide improved marker sequences and the diagnostic use thereof for the treatment of prostate carcinoma, resulting in particular in discrimination or exclusion of prostate inflammation and/or diabetes.

The provision of specific marker sequences permits a reliable diagnosis and stratification of patients with prostate carcinoma, in particular by means of a protein biochip.

The invention therefore also relates to the use of marker sequences for the diagnosis of prostate cancer, wherein at least one marker sequence of a cDNA selected from the group SEQ 1-246 or respectively a protein coded thereby or respectively a partial sequence or fragment thereof (hereinafter: marker sequences according to the invention) is determined on or from a patient to be examined.

It was possible to identify the marker sequences according to the invention by means of differential screening of samples from healthy test subjects with patient samples with prostate carcinoma on a protein biochip. For delimiting and discriminating inflammatory prostate disease(s) and/or diabetes, the marker sequences according to the invention are cross-checked with patient samples from inflammatory prostate diseases (e.g. all forms of prostatic hyperplasia, prostatitis) and diabetes on a protein chip. In doing so, it was possible to exclude one or a more different inflammatory prostate diseases and forms of diabetes. Only those marker sequences according to the invention that were not also simultaneously identified as marker sequences for inflammatory prostate diseases or diabetes continue to be considered as marker sequences for prostate carcinoma.

This process also advantageously permits the exclusion of comorbidity or polymorbidity, e.g. the presence of inflammatory prostate diseases or diabetes. On the other hand, false-positive marker sequences are excluded during the course of this process.

For the first time, these marker sequences according to the invention could be identified with sensitivity by means of protein biochips (see examples) hereby.

Thus for the first time the present invention provides marker sequences with which a special patient group may be determined within the group of prostate cancer patients (hereinafter “sub-group”). The sub-group, i.e. the patients within the group of patients with prostate cancer who do not have diabetes and/or inflammatory prostate disease, may be selectively identified (diagnosed) with these marker sequences. Thus in the scope of individualized medicine, it is possible to find patient sub-groups within the group of prostate cancer patients and select the suitable treatment/therapy for this sub-group, while patients with prostate cancer who are not in this sub-group are excluded from unsuitable therapies.

The subject matter of the invention is therefore a use of the marker sequences for identifying a sub-group of patients with prostate cancer, wherein the sub-group does not have any inflammatory prostate disease(s) and/or does not have diabetes, and wherein at least one marker sequence of a cDNA is selected from the group SEQ 1-246 and/or SEQ 247-452 or respectively a protein coded thereby or respectively a partial sequence or fragment thereof is determined on or from a patient to be examined.

The subject matter of the invention is therefore also the use of the marker sequences for the diagnosis of prostate carcinoma while excluding inflammatory prostate disease(s) or diabetes or polymorbidity, wherein at least one marker sequence of a cDNA is selected from the group SEQ 1-246 and/or SEQ 247-452 or respectively a protein coded thereby or respectively a partial sequence or fragment thereof on or from a patient to be examined, and wherein the marker sequence(s) was/were identified with a process, including:
a) Identification of marker sequence candidates using differential screening with protein biochips, for instance two protein biochips, from respectively a cDNA expression bank of a patient who has prostate cancer and a subject who does not have prostate cancer,
b) Validation of the marker sequence candidates by means of samples from patients who have inflammatory prostate disease(s) and/or samples from patients who have diabetes.

The samples used for validation come from patients who have one or more inflammatory prostate diseases and/or diabetes, but who do not have prostate cancer.

One embodiment of the invention relates to the use of the inventive marker sequences for the diagnosis of prostate carcinoma while excluding inflammatory prostate diseases or diabetes or polymorbidity, wherein 2 or 3, preferably 4 or 5, particularly preferably 6, 7 or 8 or more different marker sequences, for instance 10 to 20 or 30 or more different marker sequences, are determined on or from a patient to be examined.

One embodiment of the invention relates to the use of the inventive marker sequence(s) for the diagnosis of prostate carcinoma while excluding inflammatory prostate diseases or diabetes or polymorbidity, wherein at least one of the marker sequences is selected from the group SEQ ID No. 247, SEQ ID No. 250, SEQ ID No. 290 or a protein coded by SEQ ID No. 247, SEQ ID No. 250, SEQ ID No. 290 or from a partial sequence or fragment of SEQ ID No. 247, SEQ ID No. 250, SEQ ID No. 290. Furthermore preferred are therefore the corresponding marker sequences selected from the group SEQ ID No. 1, SEQ ID No. 4 and SEQ ID No. 44. or a protein coded by SEQ ID No. 1, SEQ ID No. 4, SEQ ID No. 44 or from a partial sequence or fragment of SEQ ID No. 1, SEQ ID No. 4, SEQ ID No. 44.

Furthermore preferred are the inventive marker sequences SEQ ID No. 249, SEQ ID No. 255, SEQ ID No. 271, SEQ ID No. 301, SEQ ID No. 341, wherein at least one of the marker sequences is selected from the group SEQ ID No. 249, SEQ ID No. 255, SEQ ID No. 271, SEQ ID No. 301, SEQ ID No. 341 or a protein coded by SEQ ID No. 249, SEQ ID No. 255, SEQ ID No. 271, SEQ ID No. 301, SEQ ID No. 341 or from a partial sequence or fragment of SEQ ID No. SEQ ID No. 249, SEQ ID No. 255, SEQ ID No. 271, SEQ ID No. 301, SEQ ID No. 341. Furthermore preferred are therefore the corresponding marker sequences selected from the group SEQ ID No. 3, SEQ ID No. 9, SEQ ID No. 25, SEQ ID No. 55, SEQ ID No. 95 or a protein coded by SEQ ID No. 3, SEQ ID No. 9, SEQ ID No. 25, SEQ ID No. 55, SEQ ID No. 95 or from a partial sequence or fragment of SEQ ID No. 3, SEQ ID No. 9, SEQ ID No. 25, SEQ ID No. 55, SEQ ID No. 95.

One embodiment of the invention relates to the use of the inventive marker sequence(s) for the diagnosis of prostate carcinoma while excluding inflammatory prostate diseases or diabetes or polymorbidity, wherein at least one marker sequence of a protein is selected from the group of neuro-oncological ventral antigen 2 (NOVA2), syntaxin 18 (STX18), heat shock 105 kDa/110 kDa protein 1 (HSPH1) or a nucleic acid coding therefor or a partial sequence or fragment thereof is determined on or from a patient to be examined and wherein the marker sequence(s) was/were identified with a method including the steps
a) Identification of marker sequence candidates using differential screening with protein biochips, for instance two protein biochips, from respectively a cDNA expression bank of a patient who has prostate cancer and a subject who does not have prostate cancer,
b) Validation of the marker sequence candidates by means of samples from patients who have inflammatory prostate disease(s) and/or samples from patients who have diabetes.

Another embodiment of the invention relates to the use of the inventive marker sequence(s) for the diagnosis of prostate carcinoma while excluding inflammatory prostate diseases or diabetes or polymorbidity in accordance with any of the foregoing claims, characterized in that the determination is made by means of in vitro diagnosis.

Another embodiment of the invention relates to the use of at least one marker sequence of a cDNA respectively selected from the group SEQ 1-246 and/or SEQ 247-452 or respectively a protein coded thereby or respectively from a partial sequence or fragment thereof as a diagnostic agent, wherein the marker sequence(s) was/were identified with a method including the steps
a) Identification of marker sequence candidates using differential screening with protein biochips, for instance two protein biochips, from respectively a cDNA expression bank of a patient who has prostate cancer and a subject who does not have prostate cancer,
b) Validation of the marker sequence candidates by means of samples from patients who have inflammatory prostate disease(s) and/or samples from patients who have diabetes.

The subject matter of the invention is also a method for the diagnosis of prostate carcinoma while excluding inflammatory prostate diseases or diabetes or polymorbidity, wherein

a.) at least one marker sequence of a cDNA selected from the group SEQ 1-246 and/or SEQ 247-452 or respectively a protein coded thereby or respectively from a partial sequence or fragment thereof is/are applied to a solid support and
b.) is/are brought into contact with body fluid or tissue extract of a patient and
c.) the detection of and interaction of the body fluid or tissue extract with the marker sequence(s) from a.) is carried out.

The subject matter of the invention is also a method for the stratification, in particular risk stratification, or for therapy control of a patient with prostate carcinoma, while excluding inflammatory prostate diseases or diabetes or polymorbidity, wherein at least one marker sequence of a cDNA selected from the group SEQ 1-246 and/or SEQ 247-452 or respectively a protein coded thereby or respectively a from partial sequence or fragment thereof is determined on or from a patient to be examined and wherein the marker sequence(s) was/were identified with a method including the steps
a) Identification of marker sequence candidates using differential screening with protein biochips, for instance two protein biochips, from respectively a cDNA expression bank of a patient who has prostate cancer and a subject who does not have prostate cancer,
b) Validation of the marker sequence candidates by means of samples from patients who have inflammatory prostate disease(s) and/or samples from patients who have diabetes.

The subject matter of the invention is also an assay, protein biochip comprising an arrangement containing at least one marker sequence of a cDNA respectively selected from the group SEQ 1-246 and/or SEQ 247-452 or respectively a protein coded thereby or respectively from a partial sequence or fragment thereof, characterized in that the marker sequence(s) is/are applied to a solid support and wherein the marker sequence(s) was/were identified with a method including the steps
a) Identification of marker sequence candidates using differential screening with protein biochips, for instance two protein biochips, from respectively a cDNA expression bank of a patient who has prostate cancer and a subject who does not have prostate cancer,
b) Validation of the marker sequence candidates by means of samples from patients who have inflammatory prostate disease(s) and/or samples from patients who have diabetes.

The subject matter of the invention is also diagnostic agents for the diagnosis of prostate carcinoma while excluding inflammatory prostate diseases or diabetes or polymorbidity containing at least one marker sequence respectively selected from the group SEQ 1-246 and/or SEQ 247-452 or respectively a protein coded thereby or respectively from a partial sequence or fragment thereof, wherein the marker sequence(s) was/were identified with a method including the steps
a) Identification of marker sequence candidates using differential screening with protein biochips, for instance two protein biochips, from respectively a cDNA expression bank of a patient who has prostate cancer and a subject who does not have prostate cancer,
b) Validation of the marker sequence candidates by means of samples from patients who have inflammatory prostate disease(s) and/or samples from patients who have diabetes.

The term “prostate carcinoma” includes only the indication prostate carcinoma or prostate cancer while excluding comorbidity or polymorbidity (definition e.g. according to Pschyrembel, de Gruyter, 261st edition (2007), Berlin). Any overlapping diseases of prostate inflammation or diabetes are strictly excluded in accordance with the invention.

The term “inflammatory prostate diseases” includes all forms of prostatitis up to and including chronic forms of prostatic inflammation, including prostate hyperplasia, especially benign prostate hyperplasia. In accordance with the invention this includes the presence of one or a plurality of different inflammatory prostate diseases.

Within the scope of this invention, diabetes shall be construed to mean for instance “Diabetes mellitus,” especially Type II Diabetes mellitus (insulin resistant), a chronic metabolic disease in which insulin production is disrupted in the beta cells of the islets of Langerhans in the pancreas, or in which, although insulin is present, it is not able to have the correct action at its target location, the cell membranes. The result of this impaired insulin production or action is elevated blood sugar levels (hyperglycemia). When discussing diabetes, a distinction is made between pre-diabetes, in which “impaired glucose tolerance” is not chemically detectable in the laboratory until it has reached its final stage, and actually manifested Diabetes mellitus. There is insulin resistance at the beginning of the pre-diabetic phase. Endothelial dysfunction, hyperlipoproteinemia, and hypertensive circulatory dysfunction develop nearly simultaneously.

Within the scope of this invention, diabetes shall be understood to include Type I diabetes, as well.

The invention therefore relates to those marker sequences that exclude comorbidity or polymorbidity with other indications such as prostate inflammation(s) or diabetes (in all forms).

In a further embodiment at least 2 to 5 or 10, preferably 30 to 50 marker sequences, or 50 to 100 or more marker sequences are determined on or from a patient to be examined.

In a further embodiment of the invention, the marker sequences according to the invention can likewise be combined, supplemented, or expanded with known biomarkers for this indication. Particularly preferred are likewise such markers as are disclosed in WO2010/000874.

In a preferred embodiment, the determination of the marker sequences is carried out outside the human body and the determination is carried out in an ex vivo/in vitro diagnosis.

In a further embodiment of the invention, the invention relates to the use of marker sequences as diagnostic agents, wherein at least one marker sequence of a cDNA is selected from the group SEQ 1-246 or respectively a protein coding therefor or respectively from a partial sequence or fragment thereof.

Furthermore, the invention relates to a method for the diagnosis of prostate carcinoma, wherein a.) at least one marker sequence of a cDNA selected from the group SEQ 1-246 or respectively a protein coding therefor or respectively from a partial sequence or fragment thereof is applied to a solid support and b.) is brought into contact with body fluid or tissue extract of a patient and c.) the detection of an interaction of the body fluid or tissue extract with the marker sequences from a.) is carried out.

The invention therefore likewise relates to diagnostic agents for the diagnosis of prostate carcinoma respectively selected from the group SEQ 1-246 or respectively a protein coding therefor or respectively from a partial sequence or fragment thereof.

In one particularly preferred embodiment, the marker sequences SEQ 1-18 are particularly preferred and SEQ 19-56 are preferred, wherein again lower numeric values are respectively preferred.

The detection of an interaction of this type can be carried out, for example, by a probe, in particular by an antibody.

The invention therefore likewise relates to the object of providing a diagnostic device or an assay, in particular a protein biochip, which permits a diagnosis or examination for prostate carcinoma.

Furthermore, the invention relates to a method for the stratification, in particular risk stratification and/or therapy control of a patient with a prostate carcinoma, wherein at least one marker sequence of a cDNA selected from the group SEQ 1-246 or respectively a protein coding therefor is determined on a patient to be examined.

Furthermore, the stratification of the patients with prostate carcinoma in new or established subgroups of prostate carcinoma is also covered, as well as the expedient selection of patient groups for the clinical development of novel therapeutic agents. The term therapy control likewise covers the allocation of patients to responders and non-responders regarding a therapy or the therapy course thereof.

“Diagnosis” for the purposes of this invention means the positive determination of a prostate carcinoma by means of the marker sequences according to the invention as well as the assignment of the patients to a prostate carcinoma. The term diagnosis covers medical diagnostics and examinations in this regard, in particular in vitro diagnostics and laboratory diagnostics, likewise proteomics and nucleic acid blotting. Further tests can be necessary to be sure and to exclude other diseases. The term diagnosis therefore likewise covers the differential diagnosis of prostate carcinoma by means of the marker sequences according to the invention and the prognosis of a prostate carcinoma.

“Stratification or therapy control” for the purposes of this invention means that the method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof.

In a further embodiment of the invention, the term “stratification” covers in particular the risk stratification with the prognosis of an outcome of a negative health event.

Within the scope of this invention, “patient” means any test subject—human or mammal—with the proviso that the test subject is tested for prostate carcinoma.

The term “marker sequences” for the purposes of this invention means that the cDNA or the polypeptide or protein that can be respectively obtained therefrom are significant for prostate carcinoma. For example, the cDNA or the polypeptide or protein that can be respectively obtained therefrom can exhibit an interaction with a sample, e.g. substances from the body fluid or tissue extract of a patient with inflammatory prostate diseases, prostate carcinoma (e.g., antigen (epitope)/antibody (paratope) interaction). For the purposes of the invention “wherein at least one marker sequence of a cDNA selected from the group SEQ 1-246 or respectively a protein coding therefor or respectively from a partial sequence or fragment thereof on or from a patient to be examined is determined” means that an interaction between the body fluid or tissue extract of a patient and the marker sequences according to the invention is detected. An interaction of this type is, e.g., a bond, in particular a binding substance on at least one marker sequence according to the invention or in the case of a cDNA the hybridization with a suitable substance under selected conditions, in particular stringent conditions (e.g., such as usually defined in J. Sambrook, E. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology,” Green Publishing Associates and Wiley Interscience, N. Y. (1989)). One example of stringent hybridization conditions is: hybridization in 4×SSC at 65° C. (alternatively in 50% formamide and 4×SSC at 42° C.), followed by several washing steps in 0.1×SSC at 65° C. for a total of approximately one hour. An example of less stringent hybridization conditions is hybridization in 4×SSC at 37° C., followed by several washing steps in 1×SSC at room temperature.

According to the invention, substances of this type are constituents of a body fluid, in particular blood, whole blood, blood plasma, blood serum, patient serum, urine, cerebrospinal fluid, synovial fluid, or of a tissue extract of the patient.

“Samples” from patients or subjects contain for instance body fluid, especially blood, whole blood, blood plasma, blood serum, patient serum, urine, cerebro-spinal fluid, synovial fluid, or a tissue extract from the patient or subject.

In a further embodiment of the invention, however, the marker sequences according to the invention can be present in a significantly higher or lower expression rate or concentration that indicates prostate carcinoma. The relative sick/healthy expression rates of the marker sequences for inflammatory prostate diseases, prostate carcinoma according to the invention are hereby determined by means of proteomics or nucleic acid blotting.

In a further embodiment of the invention, the marker sequences have a recognition signal that is addressed to the substance to be bound (e.g., antibody, nucleic acid). It is preferred according to the invention that for a protein the recognition signal is an epitope and/or a paratope and/or a hapten and for a cDNA is a hybridization or binding region.

The marker sequences according to the invention are the subject matter of Table A and can be clearly identified by the respectively cited database entry (also by means of the Internet: http://www.ncbi.nlm.nih.gov/) (see in Table A: accession no.).

The invention therefore also relates to the full-length sequences of the markers according to the invention, as defined in Table 1 via the known database entry according to Table A, referred to hereafter as SEQ 247-452; see also the associated sequence listing.

Therefore, the invention also comprises analogous embodiments of a SEQ 247-452 to the marker sequences SEQ 246, such as, e.g., described in the claims, since the SEQ 1-246 according to the invention in turn represent partial sequences, at least with high homology. The specific marker sequences SEQ 1-246 are preferred according to the invention, however.

Furthermore, SEQ 247-260 and SEQ 261-294 are preferred.

In a further embodiment of the invention, marker sequences are preferred that have P values less than or equal to 0.2, preferably less than or equal to 0.15, particularly preferably less than or equal to 0.1. In a first embodiment of the invention, the marker sequences SEQ ID No. 247, 250, 290, partial sequences, fragments, homologs or peptides/proteins coded thereby are preferred. These marker sequences have particularly suitable P values: SEQ ID No. 247 (P value: 0.1053), SEQ ID No. 250 (P value: 0.0310) and SEQ ID No. 290 (P value: 0.0254). The P value indicates the probability with which an alignment was found in the data base. See for instance http://www.ncbi.nlm.nih.gov/books/NBK62051/ for the definition of P value.

According to the invention, the marker sequences also cover those modifications of the cDNA sequence and the corresponding amino acid sequence as chemical modification, such as citrullination, acetylation, phosphorylation, glycosylation or poly(A) strand and other modifications known to one skilled in the art.

In a further embodiment of the invention, partial sequences or fragments of the marker sequences according to the invention are likewise comprised. In particular those partial sequences that have an identity of 99% or more, 98%, 97%, 96%, 95%, 94%, 93%, 92%, 91%, 90%, in particular 85%, 80% or 70% with the marker sequences according to the invention and are suitable for the inventive use—detecting prostate carcinoma while excluding inflammatory prostate diseases or diabetes or polymorbidity (so-called “homologs,” homolog marker sequences). Homologs may be protein or nucleic acid sequences.

Partial sequences are also sequences of the type which have 50 to 100 nucleotides, 70-120 nucleotides of a sequence of the SEQ 1-452, or peptides obtainable therefrom.

In a further embodiment, the respective marker sequence can be represented in different quantities in one more regions on a solid support. This permits a variation of the sensitivity. The regions can have respectively a totality of marker sequences, i.e., a sufficient number of different marker sequences, in particular 2 to 5 or 10 or more and optionally more nucleic acids and/or proteins, in particular biomarkers.

However, at least 96 to 25,000 (numerical) or more from different or identical marker sequences and further nucleic acids and/or proteins, in particular biomarkers are preferred. Furthermore preferred are more than 2,500, in particular preferred 10,000 or more different or identical marker sequences and optionally further nucleic acids and/or proteins, in particular biomarkers.

Another object of the invention relates to an arrangement of marker sequences containing at least one marker sequence of a cDNA selected from the group SEQ 1-452 or respectively a protein coding therefor. Preferably, the arrangement contains at least 2 to 5 or 10, preferably 30 to 50 marker sequences, or 50 to 100 or more marker sequences.

Within the scope of this invention, “arrangement” is synonymous with “array,” and if this “array” is used to identify substances on marker sequences, this is to be understood to be an “assay” or diagnostic device. In a preferred embodiment, the arrangement is designed such that the marker sequences represented on the arrangement are present in the form of a grid on a solid support. Furthermore, those arrangements are preferred that permit a high-density arrangement of protein binders and the marker sequences are spotted. Such high-density spotted arrangements are disclosed, for example, in WO 99/57311 and WO 99/57312 and can be used advantageously in a robot-supported automated high-throughput method.

Within the scope of this invention, however, the term “assay” or diagnostic device likewise comprises those embodiments of a device, such as ELISA, bead-based assay, line assay, Western Blot, immunochromatographic methods (e.g., so-called lateral flow immunoassays, or similar immunological single or multiplex detection measures. A protein biochip in terms of this invention is the systematic arrangement of proteins on a solid support.

The marker sequences of the arrangement are fixed on a solid support, but preferably spotted or immobilized even printed on, i.e. applied in a reproducible manner. One or more marker sequences can be present multiple times in the totality of all marker sequences and present in different quantities based on one spot. Furthermore, the marker sequences can be standardized on the solid support (i.e., by means of serial dilution series of, e.g., human globulins as internal calibrators for data normalization and quantitative evaluation).

The invention therefore relates to an assay or a protein biochip comprising an arrangement containing marker sequences according to the invention.

In a further embodiment, the marker sequences are present as clones. Clones of this type can be obtained, for example, by means of a cDNA expression library according to the invention (Büssow et al. 1998 (supra)). In a preferred embodiment, such expression libraries containing clones are obtained using expression vectors from a cDNA expression library comprising the cDNA marker sequences. These expression vectors preferably contain inducible promoters. The induction of the expression can be carried out, e.g., by means of an inductor, such as IPTG. Suitable expression vectors are described in Terpe et al. (Terpe T Appl Microbiol Biotechnol. 2003 January; 60(5): 523-33).

One skilled in the art is familiar with expression libraries, they can be produced according to standard works, such as Sambrook et al, “Molecular Cloning, A laboratory handbook, 2nd edition (1989), CSH press, Cold Spring Harbor, N.Y. Expression libraries are also preferred which are tissue-specific (e.g., human tissue, in particular human organs). Furthermore included according to the invention are expression libraries that can be obtained by exon-trapping. A synonym for expression library is expression bank.

Also preferred are protein biochips or corresponding expression libraries that do not exhibit any redundancy (so-called: Uniclone® library) and that may be produced, for example, according to the teachings of WO 99/57311 and WO 99/57312. These preferred Uniclone libraries have a high portion of non-defective fully expressed proteins of a cDNA expression library.

Within the context of this invention, the clones can also be, but not limited to, transformed bacteria, recombinant phages, or transformed cells from mammals, insects, fungi, yeasts, or plants.

The clones are fixed, spotted, or immobilized on a solid support.

The invention therefore relates to an arrangement wherein the marker sequences are present as clones.

Additionally, the marker sequences can be present in the respective form of a fusion protein, which contains, for example, at least one affinity epitope or tag. The tag may be one such as contains c-myc, his tag, arg tag, FLAG, alkaline phosphatase, VS tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase, or lacZ.

In all of the embodiments, the term “solid support” covers embodiments such as a filter, a membrane, a magnetic or fluorophore-labeled bead, a silica wafer, glass, metal, plastic, a chip, a target for mass spectrometry, or a matrix. However, a filter is preferred according to the invention.

As a filter, furthermore PVDF, nitrocellulose, or nylon is preferred (e.g., Immobilon P Millipore, Protran Whatman, Hybond N+ Amersham).

In another preferred embodiment of the arrangement according to the invention, the arrangement corresponds to a grid with the dimensions of a microtiter plate (8-12 wells strips, 96 wells, 384 wells, or more), a silica wafer, a chip, a target for mass spectrometry, or a matrix.

In a further embodiment, the invention relates to an assay or a protein biochip for identifying and characterizing a substance for prostate carcinoma, characterized in that an arrangement or assay according to the invention is a.) brought into contact with at least one substance to be tested and b.) a binding success is detected.

Furthermore, the invention relates to a method for identifying and characterizing a substance for prostate carcinoma, characterized in that an arrangement or assay according to the invention is a.) brought into contact with at least one substance to be tested and b.) a binding success is detected.

The substance to be tested can be any native or non-native biomolecule, a synthetic chemical molecule, a mixture, or a substance library.

After the substance to be tested contacts a marker sequence, the binding success is evaluated, which, for example, is carried out using commercially available image analyzing software (GenePix Pro (Axon Laboratories), Aida (Ray test), ScanArray (Packard Bioscience)).

The visualization of protein-protein interactions according to the invention (e.g., protein on marker sequence, as antigen/antibody) or corresponding “means for detecting the binding success” can be performed, for example, using fluorescence labeling, biotinylation, radioisotope labeling, or colloid gold or latex particle labeling in the usual way. A detection of bound antibodies is carried out with the aid of secondary antibodies, which are labeled with commercially available reporter molecules (e.g., Cy, Alexa, Dyomics, FITC, or similar fluorescent dyes, colloidal gold or latex particles), or with reporter enzymes, such as alkaline phosphatase, horseradish peroxidase, etc., and the corresponding colorimetric, fluorescent, or chemiluminescent substrates. Readout is conducted, e.g., using a microarray laser scanner, a CCD camera, or visually.

In a further embodiment, the invention relates to a drug/active substance or prodrug developed for prostate carcinoma and obtainable through the use of the assay or protein biochip according to the invention.

The invention therefore likewise relates to the use of an arrangement according to the invention or an assay for screening active substances for prostate carcinoma.

In a further embodiment, the invention therefore likewise relates to a target for the treatment and therapy of prostate carcinoma respectively selected from the group SEQ 1-246 or a protein respectively coding therefor.

In a further embodiment, the invention likewise relates to the use of the marker sequences according to the invention, preferably in the form of an arrangement, as an affinity material for carrying out an apheresis or in the broadest sense a blood lavage, wherein substances from body fluids of a patient with prostate carcinoma, such as blood or plasma, bind to the marker sequences according to the invention and consequently can be selectively withdrawn from the body fluid. Corresponding pertinent devices are known, such as e.g. chromatographic devices containing beads, balls, or chromatographic material, e.g. in a column, that have the inventive marker sequences and therefore can selectively withdraw e.g. (auto)antibodies.

### EXAMPLES AND FIGURES

Ten or more patient samples were individually screened against a cDNA expression library. The expression clones specific to prostate carcinoma were determined through a comparison with ten or more healthy samples. Further, the identified markers were cross-checked against samples from patients with inflammatory prostate disease or diabetes. False positive markers were removed. The subsequent identity of the remaining marker sequences was determined by DNA sequencing.

FIG. 1 shows the differential screening between two protein biochips from respectively one cDNA expression bank of a patient and a healthy test subject. The differential clones are detected by means of fluorescent labeling and evaluated by means of bioinformatics.

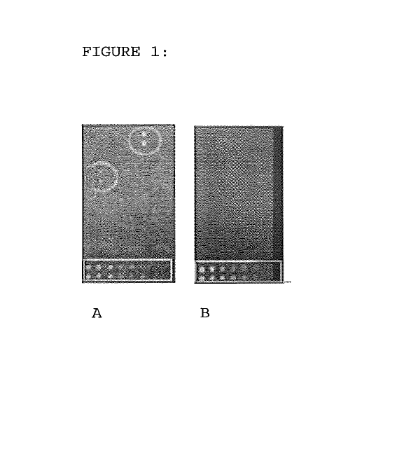

In the scope of the biomarker identification, various bioinformatic analyses are performed. For each serum, reactivities against approximately 2000 different antigens are measured by means of microarray. These data are used for a ranking of the spotted antigens with respect to their differentiation capability between healthy and diseased sera. This analysis is performed by means of the non-parameterized Mann-Whitney test on normalized intensity data. An internal standard which is also spotted on each chip is used for the normalization. Since a p value is calculated for each antigen, methods are used for correction of the multiple test. As a very conservative approach, a Bonferroni direction is performed and the less restrictive false discovery rate (FDR) according to Benjamini & Hochberg is additionally calculated.

Furthermore, the data are used for classification of the sera. Different multivariate methods are used hereby. These are methods from statistical learning methods such as support vector machines (SVM), neural networks, or classification trees, as well as a threshold value method, which is capable of both classification and also visual representation of the data.

To avoid overfitting, a 10-fold cross-validation of the data is performed.

TABLE A

| SEQ | gi | | DNA |
|---|---|---|---|
| ID | Accession | Name | Accession |
| 247 | gi\|5902723 | Neuro-oncological ventral antigen 2 (NOVA2) | NM_002516 |
| 248 | gi\|113411825 | Similar to Cyclin-L2 (Paneth cell-enhanced expression protein) | NM_030937 |
| | | transcript variant 1 (LOC727877) | |
| 249 | gi\|31543652 | Signal recognition particle 14 kDa (homologous Alu RNA binding | NM_003134 |
| | | protein) (SRP14) | |
| 250 | gi\|42544158 | Heat shock 105 kDa/110 kDa protein 1 (HSPH1) | NM_006644 |
| 251 | gi\|113428756 | Zinc finger protein 154 (pHZ-92) (ZNF154) | NM_001085384 |
| 252 | gi\|32490571 | Erythrocyte membrane protein band 4.1-like 3 (EPB41L3) | NM_012307 |
| 253 | gi\|77628146 | Endoplasmic reticulum protein 29 (ERP29) transcript variant 1 | NM_006817 |
| 254 | gi\|22749232 | Zinc finger protein 579 (ZNF579) | NM_152600 |
| 255 | gi\|194097404 | Peptide chain release factor 3 | NM_018094 |
| 256 | gi\|217330633 | Serine/Threonine kinase 36 | NM_015690 |
| 257 | gi\|16507207 | Capicua homolog (Drosophila) (CIC) | NM_015125 |
| 258 | gi\|32261293 | Protein kinase interferon-inducible double stranded RNA dependent | NM_003690 |
| | | activator (PRKRA) | |
| 259 | gi\|49574506 | Neugrin neurite outgrowth associated (NGRN) transcript variant 1 | NM_001033088 |
| 260 | gi\|89031690 | Chromosome 10 genomic contig, reference assembly | NM_005876 |
| 261 | gi\|113427652 | Alveolar soft part sarcoma chromosome region candidate 1 (ASPSCR1) | NM_024083 |
| 262 | gi\|40353201 | OTU domain containing 5 (OTUD5) | NM_017602 |
| 263 | gi\|66932901 | SREBP cleavage-activating protein (SCAP) | NM_012235 |
| 264 | gi\|33624820 | Septin 6 (SEPT6) transcript variant V | NM_145800 |
| 265 | gi\|40807365 | Dihydrouridine synthase 1-like (S. cerevisiae) (DUS1L) | NM_022156 |
| 266 | gi\|52851419 | Chromosome 6 open reading frame 153 (C6orf153) | NM_033112 |
| 267 | gi\|12597652 | 5′-nucleotidase domain containing 2 (NT5DC2) | NM_001134231 |
| 268 | gi\|113428394 | Plasticity-related gene 2 (PRG2) | XM_001129992 |
| 269 | gi\|40353728 | Ras and Rab interactor 3 (RIN3) | NM_024832 |
| 270 | gi\|55925649 | Transcription elongation factor A (SII)-like 2 (TCEAL2) | NM_080390 |
| 271 | gi\|34147459 | Coiled-coil domain containing 102A (CCDC102A) | NM_033212 |
| 272 | gi\|63003893 | Hypothetical protein LOC154467 Chromosome 6 open reading frame | NM_138493 |
| | | 129 (C6orf129) | |
| 273 | gi\|22538469 | Eomesodermin homolog (Xenopus laevis) (EOMES) | NM_005442 |
| 274 | gi\|34222261 | Tubulin, beta (TUBB) | NM_000972 |
| 275 | gi\|56676308 | Peptidylprolyl cis/trans isomerase NIMA-interacting 1 (PIN1) | NM_006221 |
| 276 | gi\|7669552 | Valosin-containing protein (VCP) | NM_007126 |
| 277 | gi\|34335231 | Creatine kinase brain (CKB) | NM_001823 |
| 278 | gi\|62414288 | Vimentin (VIM) | NM_003380 |
| 279 | gi\|71772259 | Ribosomal protein L5 (RPL5) | NM_000969 |
| 280 | gi\|223718111 | Solute carrier family 25 (mitochondrial carrier; phosphate carrier), | NM_213611 |
| | | member 3 (SLC25A3), nuclear gene encoding mitochondrial protein, | |
| | | transcript variant 3 | |
| 281 | gi\|33636763 | Ankyrin repeat domain 13B (ANKRD13B) | NM_152345 |
| 282 | gi\|38372936 | Chromatin modifying protein 2A (CHMP2A) transcript variant 1 | NM_014453 |
| 283 | gi\|134152707 | Arginine and glutamate rich 1 | NM_018011 |
| 284 | gi\|37577133 | Ubiquitin-conjugating enzyme E2M (UBC12 homolog yeast) (UBE2M) | NM_003968 |
| 285 | gi\|55925607 | Kelch-like 21 (Drosophila) (KLHL21) | NM_014851 |
| 286 | gi\|92859637 | Synaptotagmin V (SYT5) | NM_003180 |
| 287 | gi\|83776595 | CaM kinase-like vesicle-associated (CAMKV) | NM_024046 |
| 288 | gi\|14042969 | Integrin alpha FG-GAP repeat containing 3 (ITFG3) | NM_032039 |
| 289 | gi\|23111001 | V-maf musculoaponeurotic fibrosarcoma oncogene homolog F (avian) | NM_012323 |
| | | (MAFF) transcript variant 1 | |
| 290 | gi\|39725935 | Syntaxin 18 (STX18) | NM_016930 |
| 291 | gi\|194018519 | G1 to S phase transition 1 isoform 1 | NM_002094 |
| 292 | gi\|34147576 | RaP2 interacting protein 8 (RPIP8) | NM_001144825 |
| 293 | gi\|12597634 | B-cell CLL/lymphoma 11B (zinc finger protein) (BCL11B) transcript | NM_022898 |
| | | variant 2 | |
| 294 | gi\|22212941 | Ubiquitin associated protein 1 (UBAP1) | NM_016525 |
| 295 | gi\|38016910 | Stomatin (STOM) transcript variant 1 | NM_004099 |
| 296 | gi\|66879658 | ADP-ribosylation factor 1 (ARF1) transcript variant 4 | NM_001658 |
| 297 | gi\|68161512 | Cyclic AMP-regulated phosphoprotein 21 kD (ARPP-21) transcript | NM_016300 |
| | | variant 1 | |
| 298 | gi\|56181386 | STIP1 homology and U-box containing protein 1 (STUB1) | NM_005861 |
| 299 | gi\|49640010 | Tetratricopeptide repeat domain 3 (TTC3), transcript variant 2 | NM_001001894 |
| 300 | gi\|34222326 | HMP19 protein (HMP19) | NM_015980 |
| 301 | gi\|16445394 | Cadherin 18, type 2 (CDH18) | NM_004934 |
| 302 | gi\|5730103 | Thioredoxin-like 2 (TXNL2) | NM_006541 |
| 303 | gi\|113428396 | SHC (Src homology 2 domain containing) transforming protein 2 | XM_939572 |
| | | (SHC2) | |
| 304 | gi\|23111046 | Sorting nexin 5 (SNX5), transcript variant 1 | NM_152227 |
| 305 | gi\|13654275 | Queuine tRNA-ribosyltransferase 1 (tRNA-guanine transglycosylase) | NM_031209 |
| | | (QTRT1) | |
| 306 | gi\|82524843 | Multiple EGF-like-domains 6 (MEGF6) | NM_001409 |
| 307 | gi\|12232414 | Family with sequence similarity 59, member A (FAM59A) | NM_022751 |
| 308 | gi\|33286445 | Opioid growth factor receptor (OGFR) | NM_007346 |
| 309 | gi\|74048536 | Praja 1 (PJA1), transcript variant 2 | NM_001032396 |
| 310 | gi\|31652250 | Chromosome 3 open reading frame 19 (C3orf19) | NM_016474 |
| 311 | gi\|35250828 | Coatomer protein complex, subunit gamma (COPG) | NM_016128 |
| 312 | gi\|22748978 | Transmembrane protein 199 (TMEM199) | NM_152464 |
| 313 | gi\|68448524 | CD74 molecule, major histocompatibility complex, class II invariant | NM_004355 |
| | | chain (CD74), transcript variant 2 | |
| 314 | gi\|89111136 | Myosin, light chain 6B, alkali, smooth muscle and non-muscle | NM_002475 |
| | | (MYL6B) | |
| 315 | gi\|38679895 | Pleckstrin homology domain interacting protein (PHIP) | NM_017934 |
| 316 | gi\|17981697 | Cyclin-dependent kinase inhibitor 2C (p18, inhibits CDK4) (CDKN2C), | NM_001262 |
| | | transcript variant 1 | |
| 317 | gi\|149158722 | N-terminal EF-hand calcium binding protein 3 isoform 2 | NM_031232 |
| 318 | gi\|40807453 | Zinc finger protein 133 (ZNF133) | NM_003434 |
| 319 | gi\|17105395 | Ribosomal protein L29 (RPL29) | NM_000992 |
| 320 | gi\|11034854 | TMEM9 domain family, member B (TMEM9B) | NM_020644 |
| 321 | gi\|22749406 | Polymerase (RNA) I polypeptide D, 16 kDa (POLR1D), transcript | NM_152705 |
| | | variant 2 | |
| 322 | gi\|61742795 | Basal cell adhesion molecule (Lutheran blood group) (BCAM), | NM_005581 |
| | | transcript variant 1 | |
| 323 | gi\|113425940 | Similar to eukaryotic translation initiation factor 3, subunit 8, 110 kDa, | XM_001132509 |
| | | transcript variant 4 (LOC728689) | |
| 324 | gi\|34304357 | Nuclear factor of kappa light polypeptide gene enhancer in B-cells | NM_013432 |
| | | inhibitor-like 2 (NFKBIL2) | |
| 325 | gi\|4758985 | RAB11B, member RAS oncogene family (RAB11B) | NM_004218 |
| 326 | gi\|21361279 | Ts translation elongation factor, mitochondrial (TSFM) | NM_005726 |
| 327 | gi\|4503816 | Follicular lymphoma variant translocation 1 (FVT1) | NM_002035 |
| 328 | gi\|14110406 | Heterogeneous nuclear ribonucleoprotein D-like (HNRPDL), transcript | NM_031372 |
| | | variant 2 | |
| 329 | gi\|50345985 | ATP synthase, H+ transporting, mitochondrial F1 complex, beta | NM_001686 |
| | | polypeptide (ATP5B), nuclear gene encoding mitochondrial protein | |
| 330 | gi\|82534391 | Microtubule-associated protein tau (MAPT), transcript variant 4 | NM_016841 |
| 331 | gi\|50234883 | Zinc finger protein 358 (ZNF358) | NM_018083 |
| 332 | gi\|38455403 | Kruppel-like factor 1 (erythroid) (KLF1) | NM_006563 |
| 333 | gi\|55743084 | Asparagine-linked glycosylation 3 homolog (S. cerevisiae, alpha-1,3- | NM_005787 |
| | | mannosyltransferase) (ALG3) | |
| 334 | gi\|42475533 | Calsyntenin 3 (CLSTN3) | NM_014718 |
| 335 | gi\|113423957 | Huntingtin interacting protein 1 related (HIP1R) | XM_001132864 |
| 336 | gi\|4506456 | Reticulocalbin 2, EF-hand calcium binding domain (RCN2) | NM_002902 |
| 337 | gi\|22094134 | DOT1-like, histone H3 methyltransferase (S. cerevisiae) (DOT1L) | NM_032482 |
| 338 | gi\|27764862 | Solute carrier family 25 (mitochondrial carrier; adenine nucleotide | NM_001636 |
| | | translocator), member 6 (SLC25A6) | |
| 339 | gi\|46370065 | Exostoses (multiple) 1 (EXT1) | NM_000127 |
| 340 | gi\|4503528 | Eukaryotic translation initiation factor 4A, isoform 1 (EIF4A1) | NM_001416 |
| 341 | gi\|21359925 | High-mobility group 20A (HMG20A) | NM_018200 |
| 342 | gi\|33598925 | Scavenger receptor class B, member 2 (SCARB2) | NM_005506 |
| 343 | gi\|32880228 | Selenoprotein O (SELO) | NM_031454 |
| 344 | gi\|33695087 | Glycerol-3-phosphate dehydrogenase 1 (soluble) (GPD1) | NM_005276 |
| 345 | gi\|49087144 | Ribosomal protein, large, P0 (RPLP0), transcript variant 1 | NM_001002 |
| 346 | gi\|4758055 | CREB binding protein (Rubinstein-Taybi syndrome) (CREBBP) | NM_004380 |
| 347 | gi\|12232384 | COP9 constitutive photomorphogenic homolog subunit 7B | NM_022730 |
| | | (Arabidopsis) (COPS7B) | |
| 348 | gi\|90903236 | Glutathione peroxidase 4 (phospholipid hydroperoxidase) (GPX4), | NM_002085 |
| | | transcript variant 1 | |
| 349 | gi\|34335280 | Proteasome (prosome, macropain) 26S subunit, non-ATPase, 9 | NM_002813 |
| | | (PSMD9) | |
| 350 | gi\|62388867 | F-box protein 44 (FBXO44), transcript variant 4 | NM_001014765 |
| 351 | gi\|7705806 | Coenzyme Q4 homolog (S. cerevisiae) (COQ4) | NM_016035 |
| 352 | gi\|25777670 | Protein phosphatase 1, regulatory subunit 10 (PPP1R10) | NM_002714 |
| 353 | gi\|4758647 | Kinesin family member 5B (KIF5B) | NM_004521 |
| 354 | gi\|4503100 | Cysteine and glycine-rich protein 2 (CSRP2) | NM_001321 |
| 355 | gi\|54792141 | Reprimo, TP53 dependent G2 arrest mediator candidate (RPRM) | NM_019845 |
| 356 | gi\|29571103 | KiSS-1 metastasis-suppressor (KISS1) | NM_002256 |
| 357 | gi\|16306542 | Fibroblast growth factor 13 (FGF13), transcript variant 1B | NM_033642 |
| 358 | gi\|52630439 | FK506 binding protein 8, 38 kDa (FKBP8) | NM_012181 |
| 359 | gi\|21735620 | Malate dehydrogenase 2, NAD (mitochondrial) (MDH2) | NM_005918 |
| 360 | gi\|13399295 | MYC-associated zinc finger protein (purine-binding transcription | NM_002383 |
| | | factor) (MAZ) | |
| 361 | gi\|58219047 | Hairy and enhancer of split 5 (Drosophila) (HES5) | NM_001010926 |
| 362 | gi\|67906194 | Ankyrin repeat and sterile alpha motif domain containing 6 (ANKS6) | NM_173551 |
| 363 | gi\|20127628 | Zinc finger protein 768 (ZNF768) | NM_024671 |
| 364 | gi\|4758937 | Phospholipase C, beta 2 (PLCB2) | NM_004573 |
| 365 | gi\|11496989 | Poly (ADP-ribose) polymerase family, member 1 (PARP1) | NM_001618 |
| 366 | gi\|113420584 | Similar to CXYorf1-related protein, transcript variant 1 (LOC727741) | XM_001125712 |
| 367 | gi\|32484989 | WD repeat and SOCS box-containing 2 (WSB2) | NM_018639 |
| 368 | gi\|32964831 | Hypothetical protein MGC42174 (MGC42174) | NM_152383 |
| 369 | gi\|20149616 | Neural proliferation, differentiation and control, 1 (NPDC1) | NM_015392 |
| 370 | gi\|34996486 | HesB like domain containing 1 (HBLD1) | NM_194279 |
| 371 | gi\|4758219 | Family with sequence similarity 50, member A (FAM50A) | NM_004699 |
| 372 | gi\|70609878 | Ribosomal protein S2 (RPS2) | NM_002952 |
| 373 | gi\|83267865 | Dynein, light chain, LC8-type 1 (DYNLL1), transcript variant 1 | NM_001037494 |
| 374 | gi\|113422143 | Similar to 60S ribosomal protein L21, transcript variant 2 (LOC731567) | XM_001133519 |
| 375 | gi\|113430220 | CXYorf1-related protein, transcript variant 1 (LOC376475) | XM_377073 |
| 376 | gi\|17986282 | Tubulin, alpha 3 (TUBA3) | NM_006009 |
| 377 | gi\|23238232 | High mobility group nucleosomal binding domain 4 (HMGN4) | NM_006353 |
| 378 | gi\|39725675 | CDK2-associated protein 2 (CDK2AP2) | NM_005851 |
| 379 | gi\|46389548 | Endosulfine alpha (ENSA), transcript variant 3 | NM_004436 |
| 380 | gi\|46389553 | Endosulfine alpha (ENSA), transcript variant 4 | NM_207044 |
| 381 | gi\|46389549 | Endosulfine alpha (ENSA), transcript variant 1 | NM_207042 |
| 382 | gi\|47078237 | G protein pathway suppressor 1 (GPS1), transcript variant 1 | NM_212492 |
| 383 | gi\|47078280 | Family with sequence similarity 53, member B (FAM53B) | NM_014661 |
| 384 | gi\|4757715 | Sperm associated antigen 7 (SPAG7) | NM_004890 |
| 385 | gi\|50557645 | Zinc finger, FYVE domain containing 27 (ZFYVE27), transcript variant | NM_001002261 |
| | | 1 | |
| 386 | gi\|5174742 | Ubiquinol-cytochrome c reductase, Rieske iron-sulfur polypeptide 1 | NM_006003 |
| | | (UQCRFS1) | |
| 387 | gi\|53759121 | Adenomatosis polyposis coli (APC) | NM_000038 |
| 388 | gi\|57242754 | Calsyntenin 1 (CLSTN1), transcript variant 2 | NM_014944 |
| 389 | gi\|7705480 | Ubiquitin-fold modifier conjugating enzyme 1 (UFC1) | NM_016406 |
| 390 | gi\|82659090 | Staufen, RNA binding protein, homolog 1 (Drosophila) (STAU1), | NM_001037328 |
| | | transcript variant T5 | |
| 391 | gi\|83641890 | Glyceraldehyde-3-phosphate dehydrogenase (GAPDH) | NM_002046 |
| 392 | gi\|57222567 | Myeloid/lymphoid or mixed-lineage leukemia (trithorax homolog, | NM_005937 |
| | | Drosophila); translocated to, 6 (MLLT6) | |
| 393 | gi\|14249519 | Hypothetical protein FLJ14668 (FLJ14668) | NM_032822 |
| 394 | gi\|38201669 | Inhibitor of growth family, member 4 (ING4) | NM_016162 |
| 395 | gi\|83656775 | Eukaryotic translation elongation factor 2 (EEF2) | NM_001961 |
| 396 | gi\|20127557 | Chromatin modifying protein 5 (CHMP5) | NM_016410 |
| 397 | gi\|20302149 | Lysophospholipase II (LYPLA2) | NM_007260 |
| 398 | gi\|23238257 | Carnitine palmitoyltransferase 1B (muscle) (CPT1B), | NM_152247 |
| | | nuclear gene encoding mitochondrial protein, transcript variant 4 | |
| 399 | gi\|33286421 | Pyruvate kinase, muscle (PKM2), transcript variant 3 | NM_182471 |
| 400 | gi\|34147626 | Zinc finger protein 447 (ZNF447) | NM_023926 |
| 401 | gi\|38176162 | Ring finger protein 130 (RNF130) | NM_018434 |
| 402 | gi\|47933378 | N-ethylmaleimide-sensitive factor attachment protein, alpha (NAPA) | NM_003827 |
| 403 | gi\|49472815 | Fascin homolog 1, actin-bundling protein (Strongylocentrotus | NM_003088 |
| | | purpuratus) (FSCN1) | |
| 404 | gi\|67189547 | Ribosomal protein L6 (RPL6), transcript variant 2 | NM_000970 |
| 405 | gi\|6912539 | Nucleotide binding protein 2 (MinD homolog, E. coli) (NUBP2) | NM_012225 |
| 406 | gi\|73622128 | Caspase 6, apoptosis-related cysteine peptidase (CASP6), transcript | NM_001226 |
| | | variant alpha | |
| 407 | gi\|83641894 | Heterogeneous nuclear ribonucleoprotein A1 (HNRPA1), transcript | NM_031157 |
| | | variant 2 | |
| 408 | gi\|94721349 | Islet cell autoantigen 1, 69 kDa (ICA1), transcript variant 2 | NM_004968 |
| 409 | gi\|42794610 | 6-phosphogluconolactonase (PGLS), | NM_012088 |
| 410 | gi\|113420239 | Similar to block of proliferation 1 (LOC727967) | XM_001126255 |
| 411 | gi\|14149778 | Chromosome 1 open reading frame 160 (C1orfl60) | NM_032125 |
| 412 | gi\|16306547 | Seryl-tRNA synthetase (SARS) | NM_006513 |
| 413 | gi\|20336240 | Proprotein convertase subtilisin/kexin type 1 inhibitor (PCSK1N) | NM_013271 |
| 414 | gi\|22027621 | TNF receptor-associated factor 4 (TRAF4), transcript variant 1 | NM_004295 |
| 415 | gi\|31543190 | Chromosome 10 open reading frame 58 (C10orf58) | NM_032333 |
| 416 | gi\|32129198 | Cytokine induced protein 29 kDa (CIP29) | NM_033082 |
| 417 | gi\|32455235 | Helicase (DNA) B (HELB) | NM_033647 |
| 418 | gi\|34452680 | Ring finger protein 10 (RNF10) | NM_014868 |
| 419 | gi\|34996518 | Galectin-3 internal gene (GALIG) | NM_194327 |
| 420 | gi\|40804743 | Sema domain, immunoglobulin domain (Ig), transmembrane domain | NM_017789 |
| | | (TM) and short cytoplasmic domain, (semaphorin) 4C (SEMA4C) | |
| 421 | gi\|41393582 | EGF-like-domain, multiple 7 (EGFL7), transcript variant 2 | NM_201446 |
| 422 | gi\|46430498 | V-rel reticuloendotheliosis viral oncogene homolog A, | NM_021975 |
| | | nuclear factor of kappa light polypeptide gene enhancer in B-cells 3, | |
| | | p65 (avian) (RELA) | |
| 423 | gi\|47519746 | Mitogen-activated protein kinase 11 (MAPK11) | NM_002751 |
| 424 | gi\|50233802 | NDRG family member 4 (NDRG4) | NM_020465 |
| 425 | gi\|56090145 | Hypothetical LOC339123 (LOC339123), | NM_001005920 |
| 426 | gi\|71164877 | Ribosomal protein S12 (RPS12) | NM_001016 |
| 427 | gi\|72534683 | Phospholipase D family, member 3 (PLD3), transcript variant 1 | NM_001031696 |
| 428 | gi\|7305502 | Stomatin (EPB72)-like 2 (STOML2) | NM_013442 |
| 429 | gi\|90193629 | Septin 5 (SEPT5) | NM_002688 |
| 430 | gi\|50592995 | Tubulin, beta 3 (TUBB3) | NM_006086 |
| 431 | gi\|16554608 | Mitochondrial ribosomal protein S11 (MRPS11), nuclear gene encoding | NM_022839 |
| | | mitochondrial protein, transcript variant 1 | |
| 432 | gi\|30581139 | Proteasome (prosome, macropain) activator subunit 1 (PA28 alpha) | NM_006263 |
| | | (PSME1), transcript variant 1 | |
| 433 | gi\|31317308 | Phosphatidylinositol-4-phosphate 5-kinase, type I, gamma (PIP5K1C) | NM_012398 |
| 434 | gi\|46048184 | Sterile alpha motif domain containing 10 (SAMD10) | NM_080621 |
| 435 | gi\|56549124 | Dynamin 2 (DNM2), transcript variant 4 | NM_001005362 |
| 436 | gi\|70166553 | GRINL1A combined protein (Gcom1), transcript variant 9 | NM_001018097 |
| 437 | gi\|40795668 | Solute carrier family 38, member 3 (SLC38A3) | NM_006841 |
| 438 | gi\|30410780 | Hypothetical protein FLJ12949 (FLJ12949), transcript variant 2 | NM_178159 |
| 439 | gi\|21361946 | Stathmin-like 4 (STMN4) | NM_030795 |
| 440 | gi\|113413590 | Similar to deoxythymidylate kinase (thymidylate kinase), transcript | XM_001126211 |
| | | variant 4 (LOC727761) | |
| 441 | gi\|113430465 | Similar to ataxin 7-like 3 (LOC392485) | XR_018762 |
| 442 | gi\|22219473 | Fas (TNFRSF6)-associated via death domain (FADD) | NM_003824 |
| 443 | gi\|71361681 | Nuclear mitotic apparatus protein 1 (NUMA1) | NM_006185 |
| 444 | gi\|20336312 | B-cell CLL/lymphoma 11A (zinc finger protein) (BCL11A), transcript | NM_138559 |
| | | variant 3 | |
| 445 | gi\|34147391 | Coiled-coil domain containing 130 (CCDC130) | NM_030818 |
| 446 | gi\|12056467 | Junction plakoglobin (JUP), transcript variant 2 | NM_021991 |
| 447 | gi\|21464122 | Methyl-CpG binding domain protein 3 (MBD3) | NM_003926 |
| 448 | gi\|42544170 | Excision repair cross-complementing rodent repair deficiency, | NM_001983 |
| | | complementation group 1 (includes overlapping antisense sequence) | |
| | | (ERCC1), transcript variant 2 | |
| 449 | gi\|83523747 | Hypothetical protein FLJ13305 (FLJ13305) | NM_032180 |
| 450 | gi\|46358416 | Glutamate receptor, metabotropic 3 (GRM3) | NM_000840 |
| 451 | gi\|39812062 | Mitochondrial ribosomal protein L28 (MRPL28), nuclear gene encoding | NM_006428 |
| | | mitochondrial protein | |
| 452 | gi\|24308369 | Nudix (nucleoside diphosphate linked moiety X)-type motif 16 | NM_152395 |
| | | (NUDT16) | |

1. A method for the diagnosis of prostate carcinoma while excluding inflammatory prostate diseases or diabetes or polymorbidity, comprising determining at least one marker sequence selected from the group SEQ 1-246 and/or SEQ 247-452 or respectively a protein coded thereby or respectively from a partial sequence or fragment thereof on or from a patient to be examined and wherein the marker sequence(s) was/were identified with a method including the
a. Identification of marker sequence candidates using differential screening with protein biochips from respectively a cDNA expression bank of a patient who has prostate cancer and a subject who does not have prostate cancer,
b. Validation of the marker sequence candidates by means of samples from patients who have inflammatory prostate disease(s) and/or samples from patients who have diabetes.

2. The method of claim 1, characterized in that 2 or 3 different marker sequences, are determined on or from a patient to be examined.

3. The method of claim 1, wherein at least one of the marker sequences is selected from the group SEQ ID No. 247, SEQ ID No. 250, SEQ ID No. 290 or a protein coded by SEQ ID No. 247, SEQ ID No. 250, SEQ ID No. 290 or from a partial sequence or fragment of SEQ ID No. 247, SEQ ID No. 250, SEQ ID No. 290, and/or wherein at least one of the marker sequences is selected from the group SEQ ID No. 249, SEQ ID No. 255, SEQ ID No. 271, SEQ ID No. 301, SEQ ID No. 341 or a protein coded by SEQ ID No. 249, SEQ ID No. 255, SEQ ID No. 271, SEQ ID No. 301, SEQ ID No. 341 or from a partial sequence or fragment of SEQ ID No. 249, SEQ ID No. 255, SEQ ID No. 271, SEQ ID No. 301, SEQ ID No. 341.

4. The method of claim 1, wherein at least one marker sequence is a protein selected from the group of neuro-oncological ventral antigen 2 (NOVA2), syntaxin 18 (STX18), heat shock 105 kDa/110 kDa protein 1 (HSPH1) or a nucleic acid coding therefor or a partial sequence or fragment thereof.

5. The method of claim 1, characterized in that the determination is made by means of in vitro diagnosis.

6. The method of claim 1, characterized in that the marker sequence(s) is/are applied to a solid carrier.

7. The method of claim 6, wherein said solid carrier is a filter, a membrane, a magnetic or fluorophore-labeled bead, a silica wafer, glass, metal, plastic, a chip, a target for mass spectrometry, or a matrix.

8. The method of claim 1, wherein said determining step comprises
a.) applying at least one marker sequence selected from the group SEQ 1-246 and/or SEQ 247-452 or respectively a protein coded thereby or respectively from a partial sequence or fragment thereof is/are applied to a solid support and
b.) contacting said solid support of a) with body fluid or tissue extract of a patient and
c.) detecting an interaction of the body fluid or tissue extract with the marker sequence(s) from a.).

9. A method for risk stratification, or for therapy control of a patient with prostate carcinoma, while excluding inflammatory prostate diseases or diabetes or polymorbidity, comprising determining at least one marker sequence selected from the group SEQ 1-246 and/or SEQ 247-452 or respectively a protein coded thereby or respectively a from partial sequence or fragment thereof on or from a patient to be examined.

10. The method in accordance with claim 9, wherein the stratification or therapy control includes decisions regarding treatment and therapy of the patient, in particular hospitalization of the patient, the use, effect and/or dosage of one or more drugs, a therapeutic measure or the monitoring of a course of the disease and the course of therapy or etiology or classification of a disease, including prognosis.

11. Assay, protein biochip comprising an arrangement containing at least one marker sequence of a cDNA respectively selected from the group SEQ 1-246 and/or SEQ 247-452 or respectively a protein coded thereby or respectively from a partial sequence or fragment thereof, characterized in that the marker sequence(s) is/are applied to a solid support and wherein the marker sequence(s) was/were identified with a method including the steps
a) Identification of marker sequence candidates using differential screening with protein biochips from respectively a cDNA expression bank of a patient who has prostate cancer and a subject who does not have prostate cancer,
b) Validation of the marker sequence candidates by means of samples from patients who have inflammatory prostate disease(s) and/or samples from patients who have diabetes.
